(19) United States
(12) Patent Application Publication
(10) Pub. No.: US 2016/0051468 A1
(43) Pub. Date: Feb. 25, 2016

(54) Compositions for Prevention and/or Treatment of Gastrointestinal Imbalances in Digestive Disorders

(71) Applicant: BRIGHT DRINKS INC., Montreal (CA)

(72) Inventor: Donald L. Simmons, Dollard des Ormeaux, Quebec (CA)

(21) Appl. No.: 14/784,062

(22) PCT Filed: Apr. 11, 2014
(86) PCT No.: PCT/CA2014/000344

§ 371 Date: Oct. 13, 2015

Related U.S. Application Data
(60) Provisional application No. 61/810,903, filed on Apr. 11, 2013.

(51) Int. Cl. A61K 9/00; A61K 31/353; A61K 36/82; A23L 1/30; A23L 2/56; A23L 2/60; A23L 2/39; A61K 31/702; A23L 2/52

(52) U.S. Cl. CPC A61K 9/0095; A61K 31/702; A61K 31/353; A61K 36/82; A23L 2/52; A23L 2/56; A23L 2/60; A23L 2/39; A23L 1/3002; A23V 2002/00

(57) ABSTRACT

Compositions comprising a combination of epigallocatechin gallate (EGCG) and non-digestible oligosaccharides are described to prevent and/or treat one or more gastrointestinal (GI) imbalances associated with digestive disorders.

### CROSS-REFERENCE TO RELATED APPLICATIONS

The present patent application claims the benefits of priority of U.S. Provisional Patent Application No. 61/810,903, entitled “Compositions for Prevention and/or Treatment of Gastrointestinal Imbalances in Digestive Disorders”, and filed at the United States Patent and Trademark Office on Apr. 11, 2013, the content of which is incorporated herein by reference.

### FIELD OF THE INVENTION

The present invention generally relates to oral compositions for preventing and/or treating various gastrointestinal (GI) imbalances associated with digestive disorders, including but not limited to, imbalances in GI microflora, bowel regularities, proinflammatory cytokines and oxidative stress associated with various digestive disorders.

### BACKGROUND OF THE INVENTION

An NIH report in 2004 entitled “The Burden of Digestive Diseases in the United States” estimated the total cost of serious digestive diseases in the US, including direct and indirect costs was $141 billion and this cost is expected to be substantially higher today. Irritable bowel syndrome (IBS) affects between 10-25% of people globally with women being the major contributor. Gastroesophageal reflux disease (GERD) is experienced daily by 5-7% of people globally, 10-20% of Americans and is one of the most costly diseases reaching $12.6 billion. Inflammatory bowel diseases (IBD) represented by ulcerative colitis (UC) and Crohn's disease (CD) totaled $2 billion. The total cost for colorectal cancer was $9.5 billion and for all digestive cancers $24 billion. For peptic ulcers, the cost was $3.1 billion. Some additional less serious ailments, e.g. heartburn, not reported in the NIH study where 60 million Americans experience it at least once a month and lesser numbers on a daily basis. More than 9.5 million Americans experience indigestion on a regular basis and over 3 million have recurrent constipation. OTC gastrointestinal remedies are estimated to be in the multi-billion-dollar business.

### Major Factors Impacting Digestive Disorders

(a). Imbalances in Intestinal Microflora: Many digestive disorders are caused by unfavourable alterations in the beneficial bacterial flora of the gastrointestinal (GI) tract. The result is a significant disruption of intestinal lining integrity due to a proliferation of pathogens such as E. coli, Clostridium perfringens, H. pylori, etc. This imbalance in microflora, or dysbiosis, is most commonly caused by the use of antibiotics that can wipe out both good and bad bacteria. Other causative factors are GI infections (e.g., traveler's diarrhea), certain drugs such as acid-suppressing medications, chronic mal-digestion, chronic constipation, stress and diet. A primary role of beneficial microflora is to help protect the gut lining. Once these microflora become imbalanced, the host's immune capabilities become compromised thus leading to more serious digestive disorders.

The most common and debilitating form of digestive disorders fall under the umbrella of inflammatory bowel disease (IBD) and include Crohn's disease (CD) and ulcerative colitis (UC). Both diseases are characterized by chronic and relapsing inflammation of the intestinal tract lining resulting in severe watery bloody diarrhea and abdominal pain. IBD is described as a group of chronic disorders of the intestinal tract characterized by a microflora imbalance and excessive production of reactive oxygen species (ROS) and proinflammatory agents, i.e., cytokines. A definite imbalance in gut microbiota composition exists in patients with IBD compared with healthy individuals. One such study (Sokol et al, Inflammatory Bowel Disease, 2009, 15: pp 1183-1189) investigated the microflora of fecal samples obtained from patients with active disease (A-CD and A-UC), infective colitis (IC), patients in remission (R-CD and R-UC) and healthy subjects (HS). The results demonstrated that the anti-inflammatory commensal beneficial species Faecalibacterium prausnitzii (FP) along with bifidobacteria species were significantly diminished in active disease and colitis patients compared to healthy subjects.

In a separate study Willing et al (Inflammatory Bowel Disease, 2009, 15: pp 653-660) used biopsy samples collected from mucosal surface during colonoscopy instead of fecal sampling. The authors demonstrated that individuals with ileal CD had significantly lower numbers of FP and increased numbers of E coli compared to healthy subjects and those with colonic CD. The simultaneous presence of pathogens such as Clostridium spp and E coli and the absence of FP are 100 times more likely to be found in CD patients than in healthy people (Martinez-Medina M et al, Inflammatory Bowel Disease, 2006, 12: pp 1136-1145). Individuals with ileal CD had significantly lower numbers of FP (P<0.001) and increased numbers of E coli (P<0.03) compared to healthy subjects and those with colonic CD.

Irritable bowel syndrome (IBS) is one of the most common GI disorders in the United States and people suffering from this disorder experience alternating episodes of both constipation and diarrhea. The intestinal microflora of 25 IBS patients and gender-matched healthy volunteers were compared (Si J M et al, World Journal of Gastroenterology, 2004, 10(12):1802-1805) by monitoring fecal samples for Lactobacillus, Bifidobacterium, Bacteroides, C. perfringens, Enterobacteriaceae and Enteroccocus. Compared with the control group, IBS patients showed a significant decrease in Bifidobacterium numbers for the IBS patients but a significant increase in both Enterobacteriaceae and C. perfringens. There were no significant differences in the other microflora between the two groups. Both fecal and mucosal brush samples were used to demonstrate a 2-fold decrease in the level of bifidobacteria in IBS patients compared to healthy subjects (Kerckhoffs et al, World Journal of Gastroenterology, 2009, 15: pp. 2887-2892).

Similar imbalances have been reported in adolescent celiac patients versus healthy counterparts (Nadal et al, Journal of Medical Microbiology, 2007, 56: pp 1669-1674). In this study the ratio of Lactobacillus-Bifidobacterium to Bacteroides-E. coli was significantly reduced in celiac patients with either active or inactive disease compared with controls.

Gastric and peptic ulcers are sores that develop on the lining of the stomach and duodenum, i.e., the proximal region of the small intestine. A pathogenic bacterium called Helicobacter pylori (H. pylon) is a prime contributor to ulcer formation but non-steroidal anti-inflammatory drugs (NSAIDs) such as aspirin and ibuprofen are also major factors.

A prime factor therefore in considering intervention treatment to help treat and prevent this microflora imbalance favoring pathogens is to introduce special ingredients into a formulation capable of significantly increasing the numbers of beneficial microflora while suppressing growth of their non-beneficial counterparts.

(b). Imbalance in Inflammatory Mediators: In the healthy gut, the mucosal immune system ensures a balance between pro- and anti-inflammatory mediators. In inflammatory digestive disorders this immunological balance is severely impaired and is shifted towards the proinflammatory side. Recent investigations into possible mechanisms responsible for anti-inflammatory and anticancer effects have focused on inhibiting the activity of nuclear factor kappa beta (NE-κB). This protein complex acts as a switch to turn inflammation on and off in the body and remains sequestered in the cytoplasm of almost all cell types by an inhibitory protein kappa beta (IκB) until stimulated into action by reactive oxygen species and bacterial antigens. NF-κB plays a key role in regulating the immune response to infection but with incorrect regulation it has been linked to inflammatory diseases, improper immune development and cancer. Once stimulated into action, NF-κB sets off a cascade of proinflammatory cytokines such as tumor necrosis factor alpha (TNFα), interferon gamma (IFNγ), interleukins 1 and 8 (IL-1, IL-8) and enzymes like nitric oxide synthase.

The activation of NF-κB can play a critical role in the development and perpetuation of many inflammatory disorders such as IBD and infectious colitis. Several groups have reported on increased intestinal NE-κB activation in patients with IBD as well as in drug-induced IBD in animal models. Consequently the NF-κB pathway is an attractive target for therapeutic interventions. Many of the established immunosuppressive drugs used in inflammatory bowel disorders like corticosteroids, sulfasalazine and methotrexate are known to mediate their anti-inflammatory effects in part via inhibition of NF-κB activity but are beset with side effects. Inclusion of all-natural bioactive ingredients with the potential to inhibit over-expression of NE-κB and proinflammatory cytokines would be a welcome addition to health professionals in helping to alleviate the inflammatory responses prominent in many digestive disorders.

(c). Imbalance in Oxidative Stress: Oxidative stress arises when there is a marked imbalance between the production of reactive oxygen species (ROS) and their removal by antioxidants. In reaction to mild oxidative stress, tissues often respond by producing more antioxidants; however, severe persistent oxidative stress depletes body antioxidant resources and overtakes its ability to produce more antioxidants, leading to lower antioxidant levels and injury in the tissues.

ROS arise from superoxide anions released in substantial numbers by a commensal intestinal microflora called Enterococcus faecalis (Huycke et al, Carcinogenesis, 2002, 23(3): pp 529-36). Superoxide anions in the mildly acidic environment of the large intestine spontaneously disproportionate into hydrogen peroxide and oxygen. In excess however, and especially in the presence of trace elements found in diets, these superoxide anions and hydrogen peroxide can be readily converted into carcinogenic hydroxyl radicals that can cause significant damage to DNA.

The effect of certain dietary ingredients in generating excess ROS has been demonstrated by Erhardt et al. (Journal Nutrition, 1997, 127: pp 706-09). Two different diets were administered to healthy volunteers over 12 day periods with a 1-week washout period between the two treatment periods. Diet 1 was low in fiber and high in fat & red meat, diet 2 low in fat and high in fiber. Fecal samples were collected at the end of each period and analyzed for harmful hydroxyl radicals. The results demonstrated a 13-fold increase in hydroxyl radical formation in diet 1 and a corresponding significant increase in iron content. This hydroxyl radical increase arises from the catalytic-promoted reaction of trace iron in red meat from diet 1 with superoxide anion. Therefore the inclusion of a bioactive ingredient with the potential to neutralize or scavenge excess ROS is also an essential requirement for the formulation by helping to inhibit the release of proinflammatories and their resulting impact on abdominal discomfort.

### EGCG and NDO Roles in Digestive Disorders

(a). Restoration of Beneficial GI Microflora Balance & Bowel Regularity: The beneficial effects of green tea catechins on fecal microflora were demonstrated in a study involving 15 elderly subjects (average age 70.3 years) in a long-term care facility (Goto et al, 1998, Annals of Long-Term Care, 6(2):43). All subjects received the same daily diet of gastroenteral liquid supplemented with 100 mg green tea catechins immediately before each meal (daily catechin intake 300mg=˜100 mg EGCG). The protocol was continued for a period of 21 days at which time the supplement was discontinued for a 6-day period. Fecal samples were collected at 0, 7, 14, 21 and 27 days for fecal microbial analyses. The results demonstrated significant increases in bifidobacteria numbers at 7, 14 and 21 days, in lactobacilli numbers at 21 days and both microflora numbers returning to baseline values at 27 days. Significant decreases were also observed in putrefactive bacteria such as Bacteroidaceae, Enterobacteriaceae, and lecithinase-negative clostridia.

The effect of tea catechins on fecal conditions of elderly residents in a long-term care facility was investigated by the same group as in the previous study (Goto et al, 1999, Journal of Nutritional Science and Vitaminology, 45:135-141). Thirty-five residents were maintained on the same diet and given tea catechins (300 mg) in 3 divided daily doses over a six-week period. Fecal specimens were collected at the beginning and end of the study. Final results versus those at the beginning of the study demonstrated significant reduction of fecal parameters as pH, ammonia, and sulfide along with bowel regularity improvement in those individuals tending to be constipated and diarrheal.

A major contributor to colonic pathogen inhibition is undoubtedly due to green tea catechin fermentation products. In an in vitro study (Lee et al, 2006, Research in Microbiology, 157(9):876-884), the growth of pathogenic E. coli was strongly inhibited by the presence of tea fermentation products, especially phenolic acetic and propionic acids. Both lactobacilli and bifidobacteria species were only minimally affected by these products, thus demonstrating the selective antimicrobial feature of green tea polyphenols and their fermentation products.

The association between Helicobacter pylori infection and upper gastrointestinal diseases such as chronic gastritis, peptic ulceration, and gastric cancer has been widely investigated. H. pylori are sensitive to various antibiotics in vitro but clinical trials with an antibacterial agent alone and with proton pump inhibitors have mostly failed to eradicate H. pylori. In vitro and in vivo activities of tea catechins were investigated against H. pylori. The minimum-inhibitory-concentration (MIC) of the most effective catechin EGCG for 90% of 110 mostly clinical isolates of H. pylori was determined to be 32 μg per mL (Mabe et al, 1999, Antimicrobial Agents and Chemotherapy 43(7):1788-1791). Bacteriostatic and bactericidal effects were observed at concentrations equal to 2 and 4 times the MIC.

For the in vivo study the authors used pathogen-free gerbils which were allowed free access to sterilized distilled water. Each animal was fasted for 24 hours, orally inoculated with a suspension of H. pylori and kept without sterilized food and water for 4 hours. Six weeks after inoculation gerbils (3×10 groups) were treated for two weeks as follows: (i) control without catechins; (ii) 1% catechin-containing diet (32% EGCG), and (iii) 1% catechin-containing diet plus 0.5% catechin-containing water. After 24 hour fasting the animals were sacrificed and their stomachs excised and scored by macroscopic examination for hemorrhage and gastric mucosal injury. The results showed significantly reduced scores at all dosage levels with H pylori eradication occurring in thirty-six (36) percent of the animals versus zero percent on control diets.

Numerous studies have been performed in healthy human volunteers demonstrating the significant enhancing effect of daily NDO administration on increasing numbers of beneficial GI microflora. For example, the effect of short-chain fructooligosaccharides (scFOS) intake (1, 3 & 5 g/day) on the intestinal microflora was studied in 27 healthy volunteers (Togunaga et al, 1993, Bifidus 6:143-150). Testing was conducted for six weeks divided into 3 periods: (i) 2 weeks before scFOS intake, (ii) 2 weeks during scFOS intake and (iii) 2 weeks after scFOS intake. Fecal sample results demonstrated a significant increase in bifidobacteria numbers for scFOS intake period at all three dose levels with all returning to base line values after 2 weeks removed from treatment.

The effect of ingesting scFOS (5×600 mg) or matching placebo in the form of chewable tablets on volunteers presenting with mild to moderate constipation has been demonstrated in a placebo-controlled study (Tominaga et al, Bioscience Microflora, 1999, 18(1): pp 49-53). The results showed a significant increase in both bowel movement frequency and greater fecal biomass for the scFOS treatment versus placebo. The results of administering 2 to 5 grams scFOS or placebo daily to children suffering from acute diarrhea (Juffrie, Bioscience Microflora, 2002, 21(1): pp 31-34) showed a significant reduction in the duration of hospital stay favoring scFOS versus placebo.

A double-blind, placebo-controlled study (Paineau et al, 2008, British Journal of Nutrition, 99:311-318) was performed in the medical departments of five hospitals to study the effects of regular consumption of scFOS on the digestive comfort of subjects with minor functional bowel disorders (FBD). A total of 105 subjects agreed to take part in the study and asked to complete a questionnaire related to symptoms such as abdominal discomfort or pain;

abdominal fullness, bloating or swelling; feeling of incomplete bowel movement; urgency, i.e. an imperious urge to pass stool; straining at stool. Subjects also indicated intensity on a scale from 1 to 10 (10 being the maximum intensity level). The results demonstrated that 6-week consumption of 2×2.5 g scFOS per day led to a decrease in the intensity of digestive disorders and abdominal pain in which the latter was significantly lower compared with placebo. Improvement was also noted in digestive comfort in subjects with minor FBD, thereby improving quality of life as well as social performance.

Similar dose response studies have been conducted with inulin samples of varying degrees of polymerization (DP). One study (Kolida et al, 2007, European Journal of Clinical Nutrition, 61:1189-1195) evaluated the bifidogenic efficacy of 5 and 8 grams oligofructose-enriched inulin daily during a 2-week intervention trial in healthy human adults in a double-blind, placebo-controlled, crossover human study. The results showed that bifidobacteria levels increased significantly upon ingestion of both low and high doses compared to placebo. The effect of oral ingestion of inulin (2×5 grams daily) on stimulating Bifidobacterium adolescentis (BA) and Faecalibacterium prausnitzii (FP) in 12 healthy volunteers was investigated by Ramirez-Farias et al (2008, British Journal of Nutrition 101:41-55). Fecal samples were analyzed by real-time PCR and the results showed significant increases for both BA and FP.

Numerous clinical studies have been published on oligofructose (OF) and oligofructose-enriched inulin usage in digestive disorders, especially inflammatory bowel diseases. Most studies were performed with and without probiotics and other variables such as different prebiotics and dosages. Population criteria also differed with some trials enrolling patients with active disease and others studying maintenance of remission induced by antibiotics, conventional drug treatment or surgery. Most studies enrolled small numbers of patients, which had limited statistical power and little relevance given the high placebo response rates seen in clinical trials of IBD. Finally, no study has included details of the patients' diet, which could exert a marked influence on efficacy of the various therapies. The major factors contributing to any successful improvement in a patient's well-being is attributed to an improvement in beneficial microflora numbers.

(b). Inhibiting Proinflammatory Proliferation: Various ecological studies have been conducted to demonstrate the anti-inflammatory properties of EGCG using both animal models and in vitro tests demonstrating inhibition of proinflammatory cytokine proliferation. For example Yang et al (1998, Journal of Nutrition, 128:2334-2341) examined the anti-inflammatory effects of EGCG on lipopolysaccharide-mediated TNFα release from a macrophage cell line. The results showed that EGCG induced a significant decrease in TNFα protein levels in a dose-response fashion and the authors concluded that the polyphenol blocks TNF-α gene expression and protein production by inhibiting NF-κB activation. The authors conclude by suggesting that green tea polyphenols reduce inflammatory responses by attenuating NF-κB activation. In a follow-up study, the authors (Yang et al, 2001, Molecular Pharmacology 60:528-533) used human intestinal epithelial cell (IEC) lines to demonstrate the effect of EGCG in blocking NF-κB activation by inhibiting its sequestrant I-κB kinase activity. These IEC cells were selected because in inflammatory bowel disease they are potent producers of proinflammatory cytokines and because the intestinal epithelium is an initial target for therapeutic intervention.

Non-digestible oligosaccharides through their intestinal microflora fermentation processes produce short-chain fatty acids (SCFA) possessing relatively potent anti-inflammatory activities. Butyrate, one of the more prominent SCFA, plays a crucial physiological role in maintaining the health and integrity of the intestinal mucosa by regulating the balance between epithelial cell proliferation, differentiation and apoptosis. The effect of butyrate on cytokine production was monitored on colonic biopsy specimens obtained from inflamed or non-inflamed mucosa of patients with acute or inactive CD, including healthy subjects (Segain et al, Gut 2000, 47: pp 397-403). Results showed higher TNFα levels from inflamed mucosa than those from non-inflamed or normal or healthy mucosa. In the presence of butyrate, TNFα levels decreased significantly in both inflamed and non-inflamed biopsies. In a separate study the authors used mononuclear cells stimulated by bacterial lipopolysaccharide (LPS) to demonstrate NF-κB inhibition by butyrate. Tedelind et al (World Journal of Gastroenterology 2007 13(20): pp 2826-3222) demonstrated that SCFA inhibit LPS-induced TNFα release from human blood-derived neutrophils and TNFα-mediated activation of NFκB in a human colon carcinoma cell line. Propionate and butyrate were equipotent, whereas acetate was slightly less effective, at suppressing NF-κB reporter activity. Di Sabatino et al (Alimentary Pharmacology & Therapeutics, 2005, 22: pp 789-794) studied the effect of administering enteric-coated butyrate tablets (4 grams per day) to thirteen patients with mild to moderate ileocolonic Crohn's disease for 8 weeks. Among the nine patients (69%) who responded to treatment, seven (53%) achieved remission and two had a partial response. Mucosal levels of NF-κB and IL-1β were significantly decreased after treatment.

(c). Neutralizing Excess ROS Proliferation: Reactive oxygen species (ROS) such as superoxide anion, hydroxyl radical and hydrogen peroxide are common by-products of colonic bacteria activity. Each of these ROS can cause extensive damage to colonic epithelial cell DNA. Effective scavenging of superoxide anion and hydroxyl radical by tea catechins was demonstrated in an in vitro study in which EGCG was found to be the most effective of all catechins (Nanjo et al, Bioscience Biotechnology Biochemistry, 1999, 63(9): pp 1621-23). In a separate in vitro study (Anderson et al, Carcinogenesis, 2001, 22(8): pp 1189-93), EGCG was highly active in reducing the amount of oxidative damage sustained by DNA through hydroxyl radical attack. Butyrate and mixtures of SCFA were also protective in reducing hydrogen peroxide-induced DNA damage to isolated human colonocytes (Rosignoli et al, Carcinogenesis, 2001, 22(10): pp 1675-80).

A relevant prior art patent document includes an abandoned U.S. patent application (published under no. US 2009/0022852) by the present inventor. This application entitled “Liquid compositions comprising non-digestible oligosaccharides and green tea catechins” involved rigid attention to pH limits (4.8 to 5.0) and buffer capacity specifications along with substantial overages for each bioactive in order to meet 2-year label expiry dating at room temperature. The Natural Health Products Department of Health Canada ultimately approved marketing approval for a liquid formulation containing 3000 mg scFOS and 30 mg EGCG per 15 mL based on the patent application. An NPN number was assigned with the recommended use: “increases the numbers of bifidobacteria in the intestinal tract and provides antioxidants for the maintenance of good health”. In addition to the need for strict pH requirements for liquid EGCG and NDO combinations, these compositions are limited in scope in that taste acceptability of EGCG concentrations exceeding 50 mg per tablespoonful and inclusion of therapeutically effective amounts of the relatively insoluble inulin is virtually impossible. An additional marketing concern is the need for refrigeration after initial opening of the container in order to maintain EGCG integrity. A further limitation of liquid products is the inability to include probiotics in the formulation, especially bifidobacteria and lactobacillus species.

In view of the above-mentioned reports, there is a need for compositions presented in flavored or unflavored powder format for reconstitution in water or suitable beverage comprising, in combination, therapeutically effective amounts of epigallocatechin gallate (EGCG) and at least one non-digestible oligosaccharide (NDO), for helping to prevent and treat one or more gastrointestinal imbalances associated with one or more digestive disorders.

### SUMMARY OF THE INVENTION

In accordance with the principles of the present invention, oral compositions designed for the prevention and/or treatment of various gastrointestinal (GI) imbalances, associated with digestive disorders in a human being, comprise, in combination, therapeutically effective amounts of epigallocatechin gallate (EGCG) and of at least one non-digestible oligosaccharide (NDO). In typical yet non-limitative embodiments, the compositions comprise between about 100 and 800 mg of EGCG, and between of about 1 and 10 grams of NDO. In such embodiments, the concentration of EGCG is preferably but not necessarily about 300 mg and the concentration of the NDO is preferably but not necessarily about 5 grams.

In some non-limitative embodiments, the compositions may be provided in either flavored or unflavored powder format for reconstitution in water or other suitable liquids or beverages.

The compositions in accordance with the principles of the present invention are suitable to be administered to a human being, typically in drinkable form, for the prevention and/or treatment of one or more GI imbalances associated with one or more digestive disorders. Such GI imbalances may include imbalances in GI microflora, bowel regularities, proinflammatory cytokines and oxidative stress associated with various digestive disorders.

Other and further aspects and advantages of the present invention will be obvious upon an understanding of the illustrative embodiments about to be described or will be indicated in the appended claims, and various advantages not referred to herein will occur to one skilled in the art upon employment of the invention in practice.

### DETAILED DESCRIPTION OF THE INVENTION

Novel compositions for the prevention and/or treatment of various GI imbalances associated with digestive disorders will be described hereinafter. Although the invention is described in terms of specific illustrative embodiments, it is to be understood that the embodiments described herein are by way of example only and that the scope of the invention is not intended to be limited thereby.

In accordance with preferred embodiments in accordance with the principles of the present invention, there are provided oral compositions in flavored or unflavored powder format for reconstitution in water or a suitable liquid or beverage, the compositions comprising therapeutically effective amounts of epigallocatechin gallate (EGCG) and of at least one non-digestible oligosaccharide (NDO). Such compositions may be useful for helping to prevent and/or treat the following imbalances found in digestive disorders: microflora; bowel function; proinflammatory cytokines; oxidative stress, as well as helping to prevent GI-related cancers in a human being.

EGCG may be comprised in the liquid composition of the present invention individually or in combination with other catechins. Typically EGCG is obtained by extraction from the leaves of the green tea plant Camellia sinensis. More typically, the composition comprises EGCG from a decaffeinated green tea extract having an EGCG content of from about 25 to 99 percent by weight. Even more typically, the EGCG content is greater than 90 percent by weight. EGCG may also be obtained by synthesis. Typically, the composition comprises EGCG in the oral compositions at a concentration between about 100 and 800 mg, more typically between about 200 and 600 mg, and even more typically the concentration is about 300 mg.

Typically, the non-digestible oligosaccharides are chosen from xylo-oligosaccharides, soy-oligosaccharides, short-chain fructooligosaccharides (scFOS), trans-galactooligosaccharides, isomalto-oligosaccharides, inulin, oligofructose and the like, and/or mixtures thereof. Typically the non-digestible oligosaccharides (NDO) are comprised in the oral compositions of the present invention at a concentration of about 1 gram to about 10 grams, more typically between about 3 grams to about 8 grams. Typically, the non-digestible oligosaccharides are chosen from scFOS, oligofructose and inulin at a total concentration of 5 grams in order to minimize excess gas generation and permit both immediate and delayed fermentation activity throughout the large intestine.

The oral compositions in accordance with the present invention may also comprise an acceptable buffering agent mixture; a sweetening agent; and/or at least one flavor agent;

The oral compositions in accordance with the present invention are particularly useful for helping to improve GI functionality in those subjects suffering from various digestive disorders such as dyspepsia, constipation, diarrhea, GERD, irritable bowel syndrome, inflammatory bowel disease, celiac disease and peptic ulcers.

Therefore, according to another aspect of the present invention, it is provided methods to help treat and/or prevent microflora imbalances in the GI tract, bowel irregularities, proinflammatory cytokine proliferation and oxidative stress imbalances associated with digestive disorders along with helping to prevent GI-related cancers, comprising administering a composition in accordance with the present invention to a human being.

In such cases, the amount of epigallocatechin gallate and non-digestible oligosaccharides present in the compositions in accordance with the present invention is a therapeutically effective amount. A therapeutically effective amount is that amount necessary for epigallocatechin gallate and non-digestible oligosaccharides to perform their biological function in a synergistic manner (i.e. enhance or help maintain beneficial microflora in the colon, promote bowel regularity, inhibit proinflammatory cytokine proliferation, neutralize excess reactive oxygen species, and help to prevent GI-related cancers) without causing human beings overly negative effects. The exact amount of these bioactive agents to be used and administered will vary according to the following factors: their activity and/or purity, the type of condition being treated, the age and/or weight of the individual, the mode of administration, as well as the other ingredients in the composition.

The following composition powders are typically uniform in particle size distribution (for example less than #40 mesh) and blended and filled under nitrogen blanketing into tri-laminate (paper-aluminum-poly) sachets or packages. Appropriate adjustments of bioactive contents were made in the formulation to compensate for average assay results observed with the bioactives. The sachet contents are added with stirring into 4 to 6 ounces (120-180 mL) water until dissolved completely before ingestion. The examples are illustrative of the wide range of applicability of the present invention and are not intended to limit its scope.

### EXAMPLE 1

A beet sugar—stevia sweetened and mixed berry flavored powder for solution composition.

| Ingredients | Weight (gm) |
|---|---|
| Short chain fructooligosaccharides | 2.625 |
| Oligofructose-enriched inulin | 2.700 |
| Epigallocatechin gallate | 0.325 |
| Sodium citrate dihydrate | 0.648 |
| Citric acid anhydrous | 0.167 |
| Beet Fruit Sugar | 12.930 |
| Stevia (Reb A) | 0.025 |
| Cranberry Juice Powder | 0.600 |
| Mixed Berry Flavor | 0.980 |
| TOTAL | 21.000 |

### EXAMPLE 2

A sugar-free and mixed berry flavored powder for solution composition.

| Ingredients | Weight (gms) |
|---|---|
| Short-chain fructooligosaccharides | 2.625 |
| Oligofructose inulin | 2.700 |
| Epigallocatechin gallate | 0.325 |
| Sodium citrate dihydrate | 0.604 |
| Citric acid anhydrous | 0.182 |
| Erythritol | 6.000 |
| Sucralose | 0.040 |
| Cranberry Juice Powder | 0.600 |
| Mixed Berry Flavor | 0.924 |
| TOTAL | 14.000 |

### EXAMPLE 3

A fructose-stevia sweetened and tropical fruit flavored powder for solution containing 60 billion colony-forming units (cfu) each of B. lactis and L. rhamnosus.

| Ingredients | Weight (gms) |
|---|---|
| Short-chain fructooligosaccharides | 2.625 |
| Oligofructose inulin | 2.700 |
| Epigallocatechin gallate | 0.325 |
| Sodium citrate dihydrate | 0.276 |
| Citric acid anhydrous | 0.299 |
| B. lactis | ~0.008 |
| L. rhamnosus | ~0.009 |
| Fructose | 8.263 |
| Stevia (Reb-A) | 0.010 |
| Tropical fruit flavor | 0.450 |
| Beet colorant | 0.035 |
| TOTAL | 15.000 |

### EXAMPLE 4

Chronic Diarrhea: The formulation in EXAMPLE 2 was provided to a retired male teacher (age 75) to treat bowel irregularities following chemotherapy and radiation treatment to remove a small cancerous lesion located near the anal sphincter. His major distraction during the year after the combined chemo-radiation treatment was frequent diarrheal episodes with major reliance on daily intake of loperamide tablets to control the diarrhea.

After four weeks of once daily ingestion of EXAMPLE 2 before retiring in the evening, he was able to have much more normal bowel movements without having to rely on loperamide. In the four weeks he used only a total of four loperamide tablets on two occasions—two on week one of taking the powders and two on week three with the powders. Apart from those occasions when diarrhea arose, he had much more normal stool formation. Excess flatulence resulting from the cancer treatment was not further impacted by EXAMPLE 2 ingestion.

Following a week without treatment and decision to restart the powder regimen, he had a vigorous but short diarrheal episode at three AM following the first powder ingestion before retiring and had to resort to 2 loperamide tablets to control the episode. Later the following day he had a normal firm stool discharge. After 10 more days of regular daily ingestion of the reconstituted aqueous powder, his comments were “What a significant change! I have been as regular and normal as I have ever been with my lower digestive tract activity”.

### EXAMPLE 5

Severe Heartburn: The formulation in EXAMPLE 2 was provided to a retired male individual who had undergone heart bypass surgery in 2011. In addition to heart medications (Crestor, Metoprolol & Plavix) prescribed following the surgery, his physician recently (2014) decided to include ranitidine to treat recurring incidences of abdominal pain and burning sensations in the throat. These GERD-related reactions were so severe on two pre-ranitidine occasions (2012, 2013) that he needed to seek treatment at local hospitals. Ranitidine has provided him with some relief but still evidence of discomfort and pain in his abdomen.

Following week 1 of EXAMPLE 2 daily ingestion, he observed slight abdominal pain and sporadic incidences of burning sensations in the throat throughout the week. After the second week, he recorded no abdominal pain and no burning sensations in the throat or stomach. He described his present condition as “VERY GOOD” and now felt that “there is life in my body”.

While several embodiments of the invention have been described, it will be understood that the present invention is capable of further modifications, and this application is intended to cover any variations, uses, or adaptations of the invention, following in general the principles of the invention and including such departures from the present disclosure as to come within knowledge or customary practice in the art to which the invention pertains, and as may be applied to the essential features hereinbefore set forth and falling within the scope of the invention as described herein.

1. An oral composition for the prevention and/or treatment of at least one gastrointestinal imbalance associated with at least one digestive disorder in a human being, the composition comprising:
a) a therapeutic amount of epigallocatechin gallate (EGCG); and
b) a therapeutic amount of at least one non-digestible oligosaccharide (NDO).

2. A composition according to claim 1, wherein the composition comprises between about 100 mg to about 800 mg of EGCG.

3. A composition according to claim 1, wherein the composition comprises between about 200 mg to about 600 mg of EGCG.

4. A composition according to claim 1, wherein the composition comprises about 300 mg of EGCG.

5. A composition according to any of claims 1 to 4, wherein the composition comprises between about 1 gram to about 10 grams of the at least one NDO.

6. A composition according to any of claims 1 to 4, wherein the composition comprises between about 3 grams to about 8 grams of the at least one NDO.

7. A composition according to any of claims 1 to 4, wherein the composition comprises about 5 grams of the at least one NDO.

8. A composition according to any of claims 1 to 7, wherein the EGCG is derived from a decaffeinated green tea plant extract.

9. A composition according to any of claims 1 to 8, wherein the at least one NDO is xylo-oligosaccharides, soy oligosaccharides, short chain fructooligosaccharides, galacto-oligosaccharides, isomalto-oligosaccharides, inulin, oligofructose or mixtures thereof.

10. A composition according to any of claims 1 to 9, further comprising:
a) at least one flavor;
b) at least one sweetener; and/or
c) at least one buffering agent.

11. A composition according to any of claims 1 to 10, wherein the composition is provided in powdered form.

12. A package comprising therein a composition according to any of claims 1 to 11.

13. A package as claimed in claim 12, wherein the package comprises a unit dose of the composition.

14. A drinkable oral composition for the prevention and/or treatment of at least one gastrointestinal imbalance associated with at least one digestive disorder in a human being, the composition comprising:
a) a liquid;
b) a therapeutic amount of epigallocatechin gallate (EGCG); and
c) a therapeutic amount of at least one non-digestible oligosaccharide (NDO).

15. A drinkable composition according to claim 14, wherein the composition comprises between about 100 mg to about 800 mg of EGCG.

16. A drinkable composition according to claim 14, wherein the composition comprises between about 200 mg to about 600 mg of EGCG.

17. A drinkable composition according to claim 14, wherein the composition comprises about 300 mg of EGCG.

18. A drinkable composition according to any of claims 14 to 17, wherein the composition comprises between about 1 gram to about 10 grams of the at least one NDO.

19. A drinkable composition according to any of claims 14 to 17, wherein the composition comprises between about 3 grams to about 8 grams of the at least one NDO.

20. A drinkable composition according to any of claims 14 to 17, wherein the composition comprises about 5 grams of the at least one NDO.

21. A drinkable composition according to any of claims 14 to 20, wherein the EGCG is derived from a decaffeinated green tea plant extract.

22. A drinkable composition according to any of claims 14 to 21, wherein the at least one NDO is xylo-oligosaccharides, soy oligosaccharides, short chain fructooligosaccharides, galacto-oligosaccharides, isomalto-oligosaccharides, inulin, oligofructose or mixtures thereof.

23. A drinkable composition according to any of claims 14 to 22, further comprising:
a) at least one flavor;
b) at least one sweetener; and/or
c) at least one buffering agent.

24. A drinkable composition according to any of claims 14 to 23, wherein the liquid is water.

25. The use of a therapeutic amount of epigallocatechin gallate (EGCG) and of a therapeutic amount of at least one non-digestible oligosaccharide (NDO) in the manufacture of a composition suitable for the prevention and/or treatment of at least one gastrointestinal imbalance in a human being.

26. The use as claimed in claim 25, wherein the therapeutic amount of EGCG is between about 100 mg to about 800 mg.

27. The use as claimed in claim 25, wherein the therapeutic amount of EGCG is between about 200 mg to about 600 mg.

28. The use as claimed in claim 25, wherein the therapeutic amount of EGCG is about 300 mg.

29. The use as claimed in any of claims 25 to 28, wherein the therapeutic amount of the at least one NDO is between about 1 gram to about 10 grams.

30. The use as claimed in any of claims 25 to 28, wherein the therapeutic amount of the at least one NDO is between about 3 grams to about 8 grams.

31. The use as claimed in any of claims 25 to 28, wherein the therapeutic amount of the at least one NDO is about 5 grams.

32. A composition for the treatment of at least one gastrointestinal imbalance in a human being, the composition comprising:
a) a therapeutic amount of epigallocatechin gallate (EGCG); and
b) a therapeutic amount of at least one non-digestible oligosaccharide (NDO).

33. A composition according to claim 32, wherein the composition comprises between about 100 mg to about 800 mg of EGCG.

34. A composition according to claim 32, wherein the composition comprises between about 200 mg to about 600 mg of EGCG.

35. A composition according to claim 32, wherein the composition comprises about 300 mg of EGCG.

36. A composition according to any of claims 32 to 35, wherein the composition comprises between about 1 gram to about 10 grams of the at least one NDO.

37. A composition according to any of claims 32 to 35, wherein the composition comprises between about 3 grams to about 8 grams of the at least one NDO.

38. A composition according to any of claims 32 to 35, wherein the composition comprises about 5 grams of the at least one NDO.

39. A composition according to any of claims 32 to 38, wherein the EGCG is derived from a decaffeinated green tea plant extract.

40. A composition according to any of claims 32 to 39, wherein the at least one NDO is xylo-oligosaccharides, soy oligosaccharides, short chain fructooligosaccharides, galacto-oligosaccharides, isomalto-oligosaccharides, inulin, oligofructose or mixtures thereof.

41. A composition according to any of claims 32 to 40, further comprising:
a) at least one flavor;
b) at least one sweetener; and/or
c) at least one buffering agent.

42. The use of the composition of anyone of claims 1 to 11 for the treatment of at least one gastrointestinal imbalance in a human being.

43. The use of the composition of anyone of claims 14 to 24 for the treatment of at least one gastrointestinal imbalance in a human being.

44. The use of the composition of anyone of claims 32 to 41 for the treatment of at least one gastrointestinal imbalance in a human being.

45. A method of preventing and/or treating microflora imbalances associated with digestive disorders, comprising administering to a human being a composition according to any one of claims 1 to 11, 14 to 24, and 32 to 41.

46. A method of preventing and/or treating bowel irregularities associated with digestive disorders, comprising administering to a human being a composition according to any one of claims 1 to 11, 14 to 24, and 32 to 41.

47. A method of preventing and/or treating proinflammatory cytokine imbalances associated with digestive disorders, comprising administering to a human being a composition according to any one of claims 1 to 11, 14 to 24, and 32 to 41.

48. A method of preventing and/or treating oxidative stress imbalances associated with digestive disorders, comprising administering to a human being a composition according to any one of claims 1 to 11, 14 to 24, and 32 to 41.

49. A method of helping to prevent GI-related cancers, comprising administering to a human being a composition according to any one of claims 1 to 11, 14 to 24, and 32 to 41.
